(19) United States
(12) Patent Application Publication
(10) Pub. No.: US 2003/0229030 A1
(43) Pub. Date: Dec. 11, 2003

(54) Method of treating interleukin-6-mediated inflammatory diseases

(76) Inventor: Theoharis C. Theoharides, Brookline, MA (US)

Correspondence Address: Law Offices of Dr. Melvin Blecher, 4329 Van Ness St., NW, Washington, DC, 20016-5625, US

(21) Appl. No.: 10/166,088

(22) Filed: Jun. 11, 2002

(51) Int. Cl. A61K 31/7076; A61K 31/7048; A61K 31/353; A61K 31/525

(52) U.S. Cl. 514/27; 514/456; 514/47; 514/251

(57) ABSTRACT

The invention is a method of treating IL-6-mediated inflammatory diseases with flavonoid inhibitors of the production and secretion of IL-6 from human or animal mast or macrophage cells. The most effective flavonoid compounds include quercetin, kaempferol, myricetin and genistein, and these can be administered alone or in combination with S-adenosylmethionine, folic acid, interleukin-10 or a histamine-1 receptor antagonist such as azelastine.

The field of the invention is cytokine interleukin-6 (IL-6)-mediated inflammatory diseases in humans and animals. More specifically, the invention relates to the use of certain flavonoid compounds and histamine-1 receptor antagonists for treating inflammatory diseases mediated by IL-6.

### BACKGROUND

IL-6, a multifunctional cytokine, is rapidly elevated in the circulation during inflammatory, physiological or psychological stress, and is also associated with osteoporosis (Papanicolau, D., et al., Arch Int Med 128: 127 (1998)). IL-6 has been strongly implicated in the genesis of autoimmune disorders, plasma cell neoplasias, inflammatory processes of the skin (including scleroderma, psoriasis and delayed pressure urticaria, rheumatoid arthritis juvenile chronic arthritis, coronary artery disease (CAD) with or without atherosclerosis, interstitial cystitis, and congestive heart failure. Inflammation and IL-6 are specifically now thought to link to heart attacks (Taubes, G., Science 296: 242 (2002)).

Inflammation can occur in response to external (e.g., infection) or internal (e.g., cancer) factors and involves many cell types, primarily immune cells, including macrophages. Mast cells have been increasingly implicated in inflammatory processes where degranulation, as commonly seen in allergic reactions, is not observed (Theoharides, T C, J Clin Psychopharmacol. 22:103 (2002). Serotonin secreted from rat mast cells without exocytosis provided the first indication of differential release, but the physiological stimuli for such process remain unknown.

It is shown below that IL-1 induces selective secretion of IL-6, but not granule-stored tryptase, from human umbilical cord blood-derived mast cells (hCBMC). Stimulation of hCBMC and human leukemic mast cells (HMC-1) with IL-1 and TNF-α leads to a 10-fold synergistic increase in IL-6 production, still without tryptase. It is also shown below with ultrastructural immunogold localization that IL-6 is compartmentalized in 20-70 nm diameter vesicles and is excluded from the secretory granules of 1 μm diameter. These findings indicate that IL-1 induces selective release of IL-6 through a mechanism distinct from exocytosis. Selective IL-6 secretion may contribute to inflammation and mast cell differentiation.

More specifically, it is now known that: IL-6 levels are elevated in CAD and correlate withserum C-reactive protein levels. IL-6 is a primary inflammatory cytokine that promotes C-reactive protein-mediated blood vessel atherosclerosis. IL-6 plays a crucial role in the activation and differentiation of autoreactive T cells in vivo; blocking IL-6 function has been said to be an effective means of preventing autoimmune encephalomyelitis; an increase in the serum levels of IL-6 and its soluble receptor may be useful markers in rheumatoid arthritis; increased levels of soluble IL-6 receptorand of IL-6 are increased significantly compared to controls in juvenile chronic arthritis.

It is clear from this history that means for regulating (i.e., reducing) the production, and secretion of IL-6 will fill an important need in the treatment of certain autoimmune and inflammatory diseases. Autoimmunity is defined herein as an immune reaction raised against the host's own tissues.

As far as regulating the production and release from cells of IL-6, it is important to consider its known sources. IL-6 was originally identified in monocytes/macrophages, fibroblasts and endothelial cells (Papanicolaou, D., et al. (1998), above). Mast cells are abundant in cytokines, including IL-6 (Kruger-Krasagakes, S., et al., J. Invest. Dermatol. 106: 75 (1996)). Experiments with human skin biopsies showed that unstimulated mast cells do not contain preformed IL-6, but synthesis and secretion of IL-6 results after IgE-dependent stimulation, suggesting that IL-6 secreted by human mast cells potentially contributes to allergic, other immunologically mediated and nonspecific inflammatory responses (Kay, A B, New Engl. J. Med. 344:30 (2001)). Elevated serum IL-6 levels in patients with acute coronary syndrome derive from a cardiac source (likely cardiac mast cells) and are released into the coronary circulation, whereas in patients with congestive heart failure the elevated IL-6 levels represent a systemic release secondary to peripheral tissue sources (Deliargyris, E N et al., Am. J. Cardiol. 86:913(2000)). Moreover, systemic mastocytosis patients have elevated serum IL-6 levels that reflect disease severity (Theoharides, T C, Int. J. Allergy Immunol., 2002 in press).

### Mast Cell Biology

Mast cells are a normal component of the connective and mucosal tissues and play an important role in allergy and inflammation. They are localized in the connective tissues, but also in the mucosa of the bladder, gastrointestinal tract and lung, in the skin and the meninges of the brain, and in the heart. Mast cells are located there because these tissues are the main entry points for infective organisms, allergens and other noxious chemicals that trigger the body's immune response.

Mast cells derive from the bone marrow and migrate into the tissues where they synthesize and can secrete numerous vasoactive, nociceptive and inflammatory mediators, including cytokines. (Galli, S., N. Engl. J. Med. 328:257 (1993)). They are located perivascularly close to nerve endings and can be activated by a variety of neuroimmunoendocrine triggers. (Theoharides, T C, Int. J. Tissue React. 18:1 (1996)).

Mast cells are located at strategic points around capillaries and small blood vessels, where they are important in regulating the extent of constriction or dilation of the vessels including those which make up the blood-brain barrier, the protective lining of the brain which excludes toxic materials (Theoharides, T C, Life Sciences 46:607 (1990)).

Each mast cell contains up to 500 secretory granules, each storing more than 20 potent biological compounds. Mast cells secrete the contents of theses granules (i.e., degranulate) when triggered by various specific and non-specific mechanisms, such as the allergic reaction involving immunoglobulin E (IgE) and antigen (Ag), where IgE binds strongly to mast cells through its Fe receptor. The degranulation of mast cells in response to various agents is a biological consequence of the activation of one or more receptors which are located on the surface of the mast cell. The best known receptor is IgE, which is involved in allergic reactions. However, there has been recent evidence that neuropeptides, molecules released from neurons in the peripheral nervous system and brain, as well as some hormones, can also trigger mast cell degranulation. Critical among these are corticotropin-releasing hormone (CRH, otherwise referred to as corticotropin releasing factor, CRF) and structurally related urocortin secreted under stress (Theoharides, T C, J. Clin. Psychopharmacol., above) It is, therefore, clearly important to be able to block mast cell degranulation in response to various stimuli (Theoharides, ibid).

Compounds released by mast cell stimulation, collectively called mediators, include: histamine, kinins, prostaglandin D₂, tryptase and vasoactive intestinal peptide (VIP), which are vasodilatory, as well as serotonin, prostaglandin F₂-alpha and leukotrienes, which are vasoconstrictive. In addition, cytokines, histamine, kinins and prostaglandins can cause pain directly, while enzymes which destroy proteins and phospholipids can cause tissue damage directly. Finally, cytokines such as IL-6 can cause inflammation and regulate other biological responses (Galli (1993) above). Histamine, kinins, tryptase and VIP are preformed and are stored in granules; prostaglandins and cytokines are synthesized after activation of the cell and the mechanism of their secretion is not well understood. The secretion of both preformed, granule-stored and newly-synthesized mediators is hereinafter also referred to as activation. Activation is also henceforth defined as the release of any or all mediators from any or all secretory granules, vesicles or other components, whether in parallel, sequentially, differentially or selectively, or through some other means.

The compounds released by the mast cells following activation are known to cause many biological responses that are part of the overall response of the body to invasion by infective organisms, allergens or other stressful stimuli. Relevant examples of such responses are vasodilation and recruitment of inflammatory cells (e.g. leukocytes) from the circulation, tearing, nasal secretions, bronchoconstriction, itching of the skin, diarrhea or bladder pain. However, evidence is presented below that activated mast cells may also secrete without degranulation.

Once secreted, histamine, IL-6 and other mediators then bind to specific receptors on the surface of endothelial cells on vessels, immune cells, neurons or other tissues. Vasodilation and chemoattraction permits lymphocytes to leave the blood circulation and enter the tissue, where they cause additional mast cell activation and other responses. The process of activation continues, eventually involving many mast cells. It is important to note that there are no clinically available drugs capable of blocking degranulation, let alone activation in general. Anti-histamines, properly known as histamine receptor antagonists, act only after histamine is released (Theoharides, T C, Drugs 37:345 (1989)). They generally neither block the secretion of histamine or other mediators nor the action of any other mediators. Disodium cromoglycate (cromolyn) is called a “mast cell stabilizer” and is available for allergic conjunctivitus, rhinitis, asthma and food allergies, but its action is short-lived, it is only partially effective, it does not affect all mast cells and it is difficult to put in solution (Shapiro, G G et al, Pharmacotherapy 5:156 (1985)). Moreover, as will be shown below, cromolyn can not inhibit IL-6 secretion from human mast cells.

Mucosal mast cells have been implicated in irritable bowel syndrome (IBS) (Weston,A P et al, Digestive Diseases and Sciences 38:1590(1993)) where they have been increased in numbers and/or activated to various degrees. (Pang, X. et al, Urology 47:436 (1996)). Moreover, histamine and prostaglandins have been involved in gastrointestinal permeability and related diarrhea syndromes. (Castagliuolo, I et al. Am. J. Physiol. 271:884 (1996)). Mast cell activation is also implicated in interstitial cystitis, a painful condition of the bladder often associated with inflammation (Theoharides, T C et al, Urology, 57(Suppl.6A):147 2001)).

Mast cells are known for their involvement in allergic reactions and neuroinflammatory conditions that are precipitated or exacerbated by stress (Theoharides, T C et al., Int. J. Tissue React. 18:1 (1996)). Mast cells are not only a rich source of histamine, but also abundant in IL-6 (see above). Increased numbers of activated cardiac mast cells are found in ventricles, the sinusoidal node and the fibrous plaque associated with athersclerosis (Constantinidis et al. 1995, above). It is also known that coronarry inflammation may depend on activated mast cell-derived mediators (Laine et al. J. Pharm. Exp. Therap. 287:307 (1998))). Acute stress also activates cardiac mast cells, thus leading to the release of inflammatory components such as IL-6 (Pang et al. J. Pharm. Exp. Therap. 287:307 (1998)). Acute stress also causes increased serum levels of IL-6 in mice; this release was not seen in W/Wv mast cell deficient mice (Huang, M. et al., J. Neuroimmunol., in press 2002).

It is clear from this exposition that a means of inhibitng IL-6 secretion and/or activity, either by reducing its production in and secretion from mast cells or macrophages or by preventing the action of IL-6 on target cells would be of great value in treating various inflammatory diseases, such as those described above that are mediated by IL-6. As used herein, the expression “mediated by” is taken to mean any process involving IL-6 that participates in the initiation, development or exacerbation of an inflammatory disease. This has been achieved in the present invention by the use of specific flavonoids that have been shown previously by the present inventor to inhibit the degranulation of mast cells and to reduce inflammation, but without any release of IL-6. (Middleton et al., Pharm. Rev. 52:673 (2000). Although Crouvezier, S et al. Cytokine 7:13 (2000) have studied the effects of very high concentrations of certain phenolic compounds (flavonoids) in extracts of tea leaves on the production of cytokines from human leukocytes in vitro, these flavonoids had no effect on the production of IL-6, although they did increase the production of IL-10.

### SUMMARY OF THE INVENTION

The invention involves a method of treating IL-6-mediated inflammatory diseasses by inhibiting the secretion of IL-6 from mast cells or macrophages by an effective concentration of a flavonoid compound and/or a histamine-i receptor antagonist.

In one embodiment of the invention, the flavonoid compound is selected from the group consisting of quercetin, kaempferol, myricetin and genistein.

In another embodiment of the invention, as not all flavonoids have this effect on IL-6, the human mast cell culture system described herein can be used to screen for effective compounds.

In still another embodiment, the inventive method is used to treat inflammatory diseases such as allergic inflammation, autoimmune disorders, plasma cell neoplasias, inflammatory processes of the skin (including eczema, scleroderma, psoriasis, neurofibromatosis and delayed pressure urticaria), migraine, rheumatoid arthritis, juvenile chronic arthritis, coronary artery disease including unstable angina and C-Reactive Protein-mediated inflammation of blood vessels (including atherosclerosis), hypoperfusion ischemia, acute coronary syndrome and congestive heart failure, inflammatory bowel disease, multiple sclerosis, interstitial cystitis, and systemic mastocytosis.

### FIGURES

FIG. 1 shows ultrastructural immunogold localization of IL-6 in mast cells.

FIG. 2 shows the effect of anti-IgE alone, or together with quercetin, on the secretion of IL-6 from human mast cells (hCBMC).

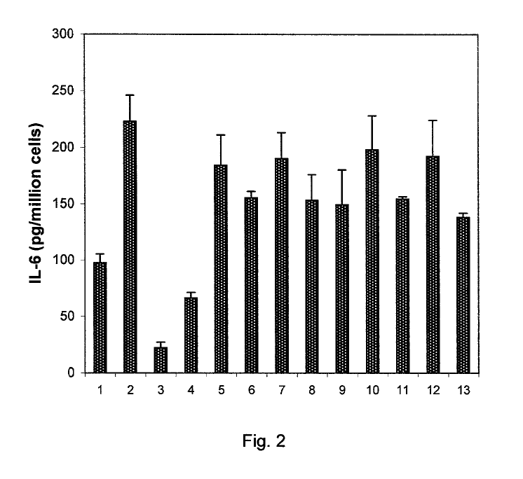

FIG. 3 shows the effects of anti-IgE, alone or together with varying concentrations of quercetin and morin or cromolyn on the release of IL-6 from human mast cells.

FIG. 4 shows the effects of anti-IgE , alone or together with two histamine-1 receptor antagonists (azelastine and olopatadine) on IL-6 secretion from hCBMC.

### DESCRIPTION OF THE PREFERRED EMBODIMENTS

It has been discovered that certain flavonoids will inhibit the production and secretion of IL-6 from human mast cells and macrophages, and that this effect provides a potential treatment of those inflammatory conditions that are mediated by or involve elevated levels of that messenger cytokine (see Background section above for a discussion of such diseases).

Flavonoids have previously been reported to inhibit mast cell secretion (Middleton, E et al, Biochemical Pharmacology 43:1167 (1992) and other inflammatory processes (see Middleton et al., 2000 above), but there was no awareness of an effect of these compounds on IL-6. Certain plant flavones (in citrus fruit pulp, seeds, sea weed) are being touted as anti-allergic, anti-inflammatory, anti-oxidant and cytoprotective with possible anti-cancer properties. I report here that only some flavones, such as quercetin, myrisetin, genistein, and kaempherol inhibit mast cell secretion of IL-6, and reverse or relieve inflammatory conditions, such as coronary artery disease, asthma, atopic dermatitis, inflammatory bowel disease, interstitial cystitis, migraines, multiple sclerosis, and rheumatoid arthritis (see expanded list above).

Quercetin inhibits secretion from human mast cells (Kimata et al. Allergy 30:501 (2000)), and has also been used effectively for the treatment of chronic prostatitis (Shoskes et al., Urology 54:960 (1999)). Other flavonoids may have opposite effects. Use of the term “bioflavonoids” or “citrus flavonoids” listed in certain commercial products, therefore, provides little information, and may include molecules that have detrimental effects. For instance, pycnogenol, marketed as an anti-inflammatory compound, actually promotes the secretion of inflammatory molecules in vitro.

The present discovery of an inhibitory effect of certain flavonoids on the production and secretion of IL-6 from human mast cells and macrophages was unexpected, as such flavonoids had previously been shown to inhibit degranulation of mast cells, and, as discussed below, I have now observed that IL-6 is not stored in mast cell granules, but rather in small (20-70 um) vesicles. Nor is it obvious to the art, as no other compound was known to inhibit IL-6 secretion from mast cells or macrophages.

It will be shown below that IL-1 induces selective secretion of IL-6, but not granule-stored tryptase, from hCBMC or hHMC-1 cells. Stimulation of HMC-l cells with IL-1 and TNF-α lead to a 10-fold synergistic increase in IL-6 production, still without tryptase arising only from degranulation that in this case does not occur. Ultrastructural immunogold localization indicates that IL-6 is compartmentalized in 20-70 nm-size vesicles and is excluded from the secretory granules. These findings indicate that IL-1 induces selective secretion of IL-6 through a mechanism distinct from degranulation.

The compositions of the invention may be formulated in any standard means of introducing pharmaceuticals orally or parenterally into a patient, e.g., by means of tablets or capsules, or administering topically by means of creams, ointments and transdermal formulations in the case of skin disease. Standard excipients and carriers for the active ingredients of the inventive compositions are described in Remington's Pharmaceutical Sciences, Mack Publishing Co., Easton, Pa.

The preferred flavonoid compounds for inhibiting the release of IL-6 from mast cell vesicles are quercetin, myrisetin, genistein and kaempferol; quercetin is highly preferred. In order to increase absorption, these flavonoids may be administered as their glycoside derivatives. These compounds may be obtained from Kaden Biochemicals, Hillsborough, N.J.

The preferred concentration range of the flavonoid components of oral formulations are 10-3,000 mg per tablet or capsule. The number of capsules or tablets to be taken per day is determined by the nature and severity of the medical condition, and is readily determinable by the patient's physician. Other representative formulations are described in the examples below.

### Exemplification

### EXAMPLE 1

### General Methods

Human cord blood-derived mast cells (HCBMC) were grown from CD34+ progenitor mononuclear umbilical cells isolated from umbilical cord blood by positive selection of AC133 (CD 133+/CD34+high) cells by magnetic cell sorting (Miltenyi Biotec, Auburn, Calif.). CD34+ cells were then cultured in Isocove's Modified Dulbecco's Medium (1MDM) containing 100 ng/ml Stem Cell Factor (from Amgen, Thousand Oaks, Calif.) plus test components in 10% FBS at 37° C. in 5% CO₂ balanced air. By 10 weeks, 95% of the cells in culture could be identified as mast cells by immuno-staining for tryptase. hCBMC were washed three times in PBS and resuspended in culture medium (1×10⁵ cells/200 ul sample). Test substances, e.g., IL-1 were added and cells were incubated at 37° C. in 5% CO₂ for six hours for dose-response experiments or for the indicated times in time-course experiments. IL-6 and tryptase were measured in cell-free supernatant fluids by ELISA (R&D Systems, Minneapolis, Minn.) and fluoroenzymeimmunoassay (Pharmacia, Uppsala, Sweden), respectively.

Human leukemic mast cells (HMC-1) was obtained from Dr. Butterfield (Mayo Clinic, Rochester, Minn.), and cultured in IMDM medium supplemented with 10% FBS, PBS and 2 μM alpha.thioglycerol. HCBMC or HMC-I cells 2×10⁵/200 μl sample were stimulated in full culture medium as indicated.

Dose-response of IL-6 and tryptase secretion from hCBMC and HMC-1 cells were tested after stimulation for six hours with indicated concentrations of IL-1 or other stimuli. Time-course of IL.6 production induced by 50 ng/ml of IL-1 a from CBMC or by 10 ng/ml IL-1 from HMC-1 cells could be tested.

The data in general represent means +/− standard error of the mean from 3 to 4 experiments done in duplicate for each cell type used.

### EXAMPLE 2

### Ultrastructural Immunocytochemistry

This technique was employed to localize IL-6. hCBMC or HMC-1 cells were fixed with 5% acrolein and embedded in LR white. Microthin sections were cut and these were mounted on grids. For IL-6 localization, grids were incubated with 6.3 μg/ml (CBMC) or 20 μg/ml (HMC-1) polyclonal rabbit antihuman IL-6 antibody (Biologicals) overnight, followed by goat anti-rabbit IgG conjugated with 10 nm gold particles (1:30, Polyscience, Warrington, Pa.).

Monoclonal mouse anti-human tryptase antibody (Chemicon, Tenecula, Calif.) at 40 μg/ml), followed by goat anti-mouse IgG conjugated with 10 nm gold particles, was used to localize tryptase.

Processed grids were stained with uranylacetate and lead citrate and viewed with a CM20 transmission electron microscope. 11-6 was localized in microvesicles (20-70 nm) and tryptase in mast cell granules. Negative controls were processed similarly, but without the primary antibody. Magnification was 17,800×.

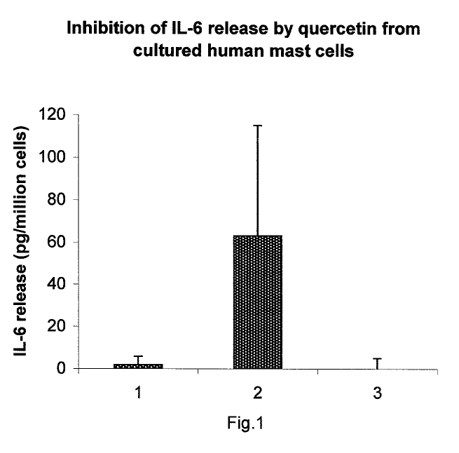

The results are shown in FIG. 1. Curved arrows indicate vescicles (about 50 nm) containing IL-6 shown by bound electron-dense gold particles. Note the clusters of gold particles (dark dots) inside (white curved arrows) and outside (dark curved arrows) of the cell (bar=50 nm).

### EXAMPLE 3

In this experiment, the effect of quercetin on the production of IL-6 and TNF-a from rat peritoneal macrophages and human mast cells that are involved in inflammation was studied. The results are shown in Table 1.

TABLE 1

Inhibition
(% of total)*

| Cell type | IL-6 | TNF-α |
|---|---|---|
| Rat macrophages** | 96.3 | 88.3 |
| Human mast cells*** | 95.8 | 78.3 |

*Quercetin dihydrate (100 μM), dissolved in 0.01% final of dimethylsulfoxide, was incubated cell suspensions 10 for mins. Prior to the stimulus, and the reaction continued for 6 hrs.
**stimulated by lipopolysaccharide (LPS)
***hCBMC stimulated by IgE and anti-IgE

The results indicated that quercetin almost completely inhibited the production of IL-6 from both cell types, and also greatly inhibited the production of TNF-A from both cell types.

### EXAMPLE 4

In this experiment, the inhibition of IL-6 secretion by quercetin from hCBMC was studied in the presence of anti-IgE, an inhibitor of the stimulating effect of IgE on production of IL-6. The results are shown in Table 2.

TABLE 2

| | | Inhibition (% of total) |
|---|---|---|
| Expt. No. | Condition | IL-6 (pg/10⁶ cells) |
| 1 | Spontaneous | 25.0 |
| 2 | ″ | 25.8 |
| 3 | ″ | 25.8 |
| 4 | Quercetin* | 10.8 |
| 5 | Anti-IgE | 105.8 |
| 6 | Anti-IgE | 150.2 |
| 7 | Quercetin* + Anti-IgE | 50.5 |

*0.1 mM for 10 mins. prior to addition of anti-IgE

The controls showed that anti-IgE antibody itself had no effect on the production and release of IL-6. However, quercetin had a profound inhibitory effect on IL-6 production and release in the presence of antibody.

### EXAMPLE 5

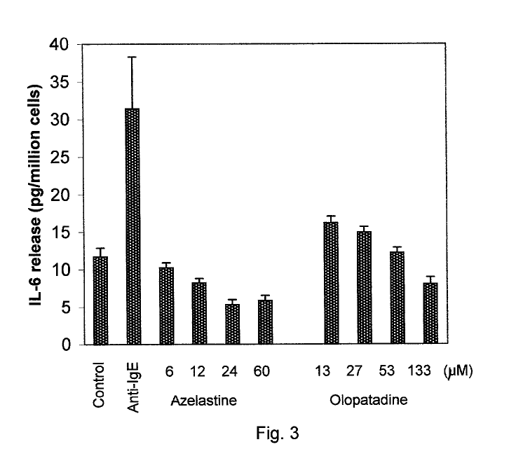

s another experiment examining the inhibition of the release of IL-6 from hCBMC by quercetin, with the results set forth graphically in FIG. 3.

In the graph, column 1 shows the spontaneous release of IL-6 from the cultured mast cells. Column 2 shows the increase in IL-6 secretion after incubation of cells with anti-IgE alone. Column 3 shows incubation of the cells with 0.1 mM quercetin for 30 mins., followed by incubation of the cells for 6 hrs. with amti-IgE. The experiment was replicated 6 times per variable.

The results, which are statistically significant, show that quercetin profoundly inhibited the secretion of IL-6 from mast cells whose production of IL-6 had been stimulated by anti-IgE antibodies.

### EXAMPLE 6

the effects of anti-IgE, alone and or after with the flavonoid n on the secretion of IL-6 from hCBMC. The compositions of FIG. 3 are shown below.

| | |
|---|---|
| Bar # | Drug and Concentration |

| | | | |
|---|---|---|---|
| 1 | Spontaneous | | |
| 2 | Anti-IgE* | | |
| 3 | Quercetin, | 100 | μM |
| 4 | ″ | 10 | μM |
| 5 | ″ | 1 | μM |
| 6 | ″ | 0.1 | μM |
| 7 | ″ | 0.01 | μM |
| 8 | Morin | 100 | μM |
| 9 | ″ | 10 | μM |
| 10 | ″ | 1 | μM |
| 11 | ″ | 0.1 | μM |
| 12 | ″ | 0.01 | μM |
| 13 | Cromolyn | 100 | μM |

*Anti-IgE was present in vessels 2-13

n=4

As anticipated, anti-IgE more than doubled the secretion of IL-6 over the spontaneous control. The two highest concentrations of quercetin greatly reduced the secretion of IL-6 compared to either the spontaneous control or the anti-IgE value alone; the three lower concentration of the flavonoid only slightly reduced the secretion of the cytokine compared to IgE alone. The two concentrations of the flavonoid morin had only a small effect on IL-6 secretion in the presence of anti-IgE; so did a high concentration of cromolyn, but this effect was not statistically significant. These results demonstrate the specificity of the flavonoid effect that is quercetin, but not morin, inhibited the anti-IgE-mediated increase in the secretion of IL-6 from Human mast cells.

### EXAMPLE 7

In this experiment, the selective secretion of IL-6 from hCBMC in the absence and presence of CRH, IL-1 or anti-IgE antibody was studied. In parallel incubations, the effect of each of these agents on the secretion of tryptase from mast cell granules was also examined. The results are shown in Table 3.

TABLE 3

| | IL-6 | Tryptase |
|---|---|---|
| | (pg/ml/5 × 10⁶ cells) | % total |
| Spontaneous | 3.8 +/− 1.3 | 5.18, 4.16 |
| CRH (5 × 10⁻⁵M) | 33.3 +/− 3.5 | 4.20, 4.26 |
| | (n = 25) | (n = 2) |
| Spontaneous | 21.2 +/− 6.6 | 4.15 +/− 1.0 |
| IL-1 (50 ng/ml) | 127.4 +/− 14.8* | 4.26 +/− 0.33 |
| | (n = 4) | (n = 3) |
| Spontaneous | 54.2 +/− 20.2 | 4.06 +/− 0.82 |
| Anti-IgE (10 μg/ml) | 110.5 +/− 28.5* | 29.3 +/− 7.1* |
| | (n = 4) | (n = 4) |

*p < 0.05, paired t-test

Low concentrations of CRH, IL-1 and anti-IgE antibody profoundly increased the production and secretion of IL-6. Neither CRH nor IL-1 increased the secretion of the marker tryptase, suggesting that neither of these agents stimulated degranulation of the cells. In contrast, anti-IgE antibody greatly increased the secretion of this marker protein.

### EXAMPLE 8

In this experiment, the effects of quercetin, alone or together with IL-1, on the production and secretion of IL-6 from human mast cells was studied. The results are shown in Table 4.

TABLE 4

| | IL-6 (pg/ml/5 × 10⁵ cells) | Inhibition (% total) |
|---|---|---|
| DMSO-Control | 95.8 | |
| Quercetin (10 μM) | 74.9 | |
| Quercetin (100 μM) | 56.1 | |
| IL-1 (50 ng/ml) + DMSO | 399.4 | |
| IL-1 (50 ng/ml) + | 300.1 | 32.7* |
| Quercetin (10 μM) | | |
| IL-1 (50 ng/ml) + | 150.4 | 82.0* |
| Quercetin (100 μM) | | |

*p < 0.05

The results indicate that neither quercetin alone northe solvent (DMSO) in which it was dissolved had a significant effect on IL-6 secretion, even though the highest concentration slightly decreased the spontaneous formation of IL-6. IL-1 alone caused a great increase in IL-6. This effect of IL-1 on IL-6 was greatly inhibited by quercetin in a dose-dependent fashion.

### EXAMPLE 9

It is known (Marshall et al. J. Clin. Invest. 97:1122 (1996)) that interleukin-10 (IL-10) can inhibit IL-6 production from rat peritoneal mast cells stimulated by lipopolysaccharide (LPS) and anti-IgE antibody, even though IL-10 did not influence histamine release. No one has examined this phenomenon in human mast cells.

The effect of IL-10 on HMC-1 leukemic cells producing IL-6, especially cells stimulated by IL-1 that induces selective release of IL-6, was studied alone or together with a flavonoid compound. The results are shown in Table 5.

TABLE 5

| Conditions | IL-6 (pg/ml/10⁵ cells) | Inhibition (% total) |
|---|---|---|
| Control | 96.8 | |
| IL-1 (50 ng/ml) | 401.7 | |
| IL-10 (2 ng/ml) | 85.6 | |
| Quercetin (10 μM) | 79.8 | |
| Quercetin + IL-1 | 299.0 | 5.37 |
| IL-10 + IL-1 | 275.9 | 31.32 |
| Quercetin + IL-10 + IL-1 | 148.7 | 62.98 |

The results of these experiments indicate that a combination of low doses of IL-10 and quercetin had a synergistic inhibitory effect on IL-6 secretion from HMC-1 cells. This combination may, therefore, be effective in the treatment of inflammatory diseases presenting with high IL-6, especially systemic mastocytosis.

### EXAMPLE 10

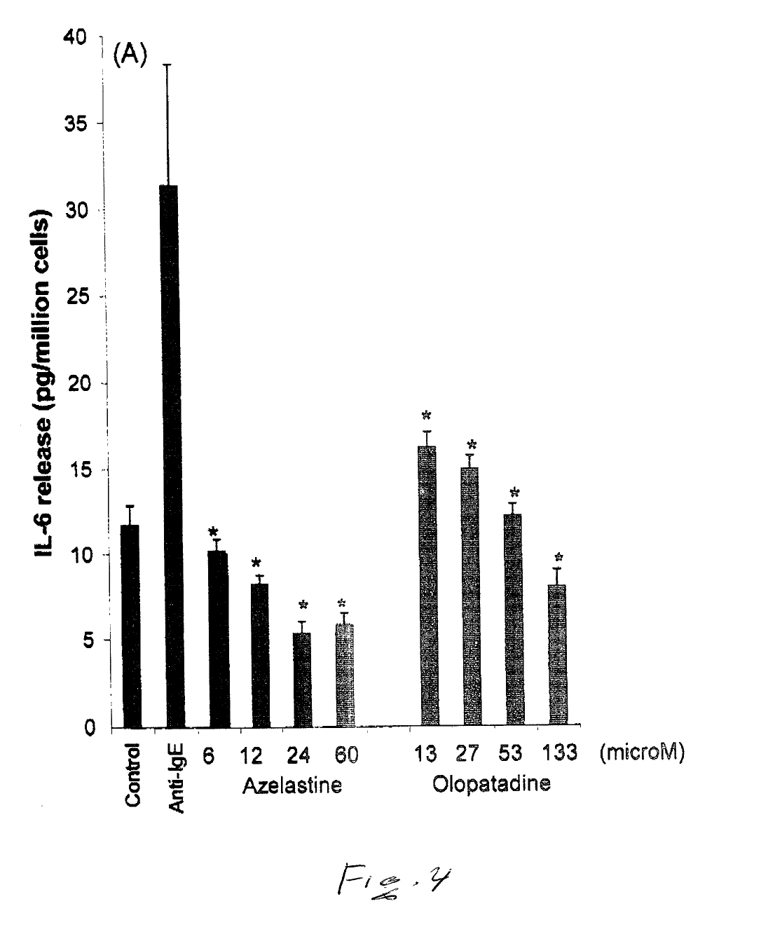

Certain histamine-i receptor antagonists have been shown to inhibit cytokine secretion from human leukemic mast cells (Lippert et al, Exp. Dermatol. 2:118 (2000). Azelastine, like olopatadine, is a histamine-1 receptor antagonist, and has been reported to inhibit tryptase secretion (Lytinas et al., Allergy Asthma Proc. 23: (2002)). Here we show (FIG. 4) that azelastine is a potent inhibitor of IL-6 secretion from hCBMC; this inhibition was dose-dependent and, at 60 μM, this compound reduced IL-6 secretion to below control levels. Azelastine.HCl may be obtained from Wallace Laboratories, Cranbury, N.J. It may also be obtained from the same company as ASTELIN, a nasal spray containing 0.1% azelastine.HCl in aqueous solution. In vivo, azelastine may be administered at a dosage of about 2 to 100 mg per 70 kg body weight per day.

At concentrations below 1 μM azelastine was ineffective; however, when added together with 10 μM quercetin, the combination inhibited IL-6 secretion from hCBMC (Table 6).

TABLE 6

| Conditions | IL-6 (pg/ml/5 × 105cells) | Inhibition (% total) |
|---|---|---|
| Control | 12.8 | |
| Anti-IgE | 31.9 | |
| Azelastine, 1 μM | 11.9 | |
| Quercetin, 10 μM | 9.8 | |
| Azelastine + Anti-IgE | 30.6 | |
| Azelastine + Quercetin + | 13.2 | 58.6 |
| Anti-IgE | | |
| Quercetin + Anti-IgE | 21.4 | 32.9 |
| Azelastine + quercetin + | 13.2 | 58.6 |
| anti-IgE | | |

These results, taken together, demonstrate that certain flavonoid compounds inhibit the production and secretion of IL-6 from human mast cells that are stimulated by different inflammatory stimuli.

I claim:

1. A method of treating interleukin-6 (IL-6)-mediated inflammatory diseases in human and animal species, comprising the step of contacting in vivo mast cells or macrophage cells from said species with a flavonoid compound in a concentration and time scale effective to inhibit the production and secretion of IL-6 from said cells.

2. The method according to claim 1, wherein said flavonoid compound is selected from the group consisting of quercetin, kaempferol, genistein, and myricetin.

3. The method according to claim 1, wherein said flavonoid compounds are administered as a glycoside derivative.

4. The method according to claim 1, wherein said effective concentration of said flavonoid compound is contained in a dose of between 10 and 3,000 mg per 70 kilogram body weight per day.

5. The method according to claim 1, wherein said effective concentration of said flavonoid compound is contained in a dose of between 40 and 200 mg per 70 kilogram body weight per day.

6. The method according to claim 1, wherein said flavonoid compound is administered together with an amount of folic acid effective to prevent homocysteine elevations that are a risk factor for coronary artery disease.

7. The method according to claim 1, wherein said flavonoid compound is administered in combination with S-adenosylmethionine in an amount sufficient to accelerate the metabolism of homocysteine to cysteine.

8. The method according to claim 1, wherein said production and secretion of said IL-6 in said cells are stimulated by Corticotropin Releasing Hormone.

9. The method according to claim 1, wherein said production and secretion of said IL-6 in said cells are stimulated by anti-IgE antibody.

10. The method according to claim 1, wherein said production and secretion of said IL-6 in said cells is stimulated by interleukin-1.

11. The method according to claim 1, wherein said flavonoid compound is administered in combination with interleukin-10 (IL-10) in an amount sufficient to inhibit IL-6 production and secretion.

12. The method according to claim 11, wherein said IL-10 is administered at a dose of between 2 and 2000 ng per 70 kg body weight per day.

13. The method according to claim 1, wherein said flavonoid compound is administered in combination with azelastine in amounts effective to inhibit the production and secretion of of IL-6.

14. The method according to claim 13, wherein said effective amount of azelastine is between 2 and 100 mg per 70 mg body weight per day.

15. The method according to any one of claims 1-14, wherein said compounds are administered in an oral or parenteral form.

16. The method according to any one of claims 1-14, where said compounds are administered in a topical form selected from the group consisting of a cream, ointment or transdermal formulation.

17. The method according to claim 1, wherein said inflammatory disease is selected from the group charactered by elevated serum and tissue levels of IL-6 in the patient, consisting of autoimmune disorders, plasma cell neoplasias, inflammatory diseases of the skin such as alopecia, eczema, scleroderma, psoriasis, neurofibramotosis, and delayed pressure urticaria, migraines, rheumatoid arthritis, juvenile chronic arthritis, unstable angina and C-Reactive Protein-mediated inflammation of blood vessels such as atherosclerosis, coronary artery disease, congestive heart failure, and reperfusion ischemia, inflammatory bowel diseases, interstitial cystitis, multiple sclerosis, asthma, and systemic mystocytosis.
